(19) United States
(12) Patent Application Publication
(10) Pub. No.: US 2009/0157115 A1
(43) Pub. Date: Jun. 18, 2009

(54) SYSTEM AND METHOD FOR RETRIEVAL OF A MEDICAL FILTER

(76) Inventor: James A. Fleming, Bethlehem, PA (US)

Correspondence Address: RONALD L. HOFER, 122 LINDBERGH LANE, MOORESVILLE, NC, 28117, US

(21) Appl. No.: 11/742,802

(22) Filed: May 1, 2007

(51) Int. Cl. A61B 17/22

(52) U.S. Cl. 606/200

(57) ABSTRACT

A medical filter retrieval system for retrieval of a medial filter from placement inside a body passage of a patient has a medical filter with a ferromagnetic head at one end thereof and a retrieval catheter. Located coaxially within the retrieval catheter are an elongated central probe having a ferromagnetic end. The medical filter is retrieved from the body passage by bringing the magnetic end of the central probe into proximity of the filter head which attracts and aligns the probe end to the filter head and magnetically attaches the probe end and head. Optionally, a snare device can be coaxially located within the retrieval catheter to engage the filter after the probe has been aligned with the filter head.

### BACKGROUND AND SUMMARY OF THE INVENTION

The present invention relates to a system and method for retrieval of a medical filter. More specifically, the present invention relates to a system and method using magnetic force for alignment and retrieval of a medical filter.

Medical filters, including vena cava filters, are emplaced inside blood vessels or other body passages for the purpose of intercepting thrombus or particles while allowing free flow of blood in the vessels or other body fluid in the body passages. Medical filters often are emplaced and never retrieved, remaining effective during their time in place, and remaining permanently in place. However, in some cases it may be desirable to remove or retrieve a medical filter after it has been in place for a period of time.

Generally speaking, it is known to use catheters to emplace or deliver medical filters in a vein or bodily passage as well as to remove medical filters following their implantation. Retrieval of medical filters can be challenging. One method of retrieval involves use of a catheter with a loop which is pushed out of the catheter and used to snare a hook on the end of a filter. One of the most difficult aspects of vascular and other medical filter retrievals is locating or aligning the correct part of the filter with the operative end or loop of the retrieval catheter. The surgeon is often working through a long catheter extending through a tortuous anatomy while viewing a shadowy image of the filter and retrieval catheter loop in a two dimensional fluoroscopy image. Fluid flow within the vein or body passage may further increase the challenge.

In light of these challenges, it would be beneficial to have an improved device and method for removal or retrieval of vascular and other medical filters. It would also be desirable to have a practical and economical system and method which facilitate removal of medical filters after implantation. It would be further desirable to have a retrieval system and method which can reduce the time required for retrieval and which can be used even under view of a shadowy, two dimensional image. Furthermore it would be desirable to have a retrieval system and method which can be used from either a femoral or jugular approach and which can be adapted for use with a wide variety of medical filter designs.

Accordingly, the present invention provides an improved system and method using magnetic force for retrieving a medical filter inside a body passage of a patient. The filter retrieval system comprises:
a medical filter having a retrieval section with a head which is ferromagnetic head at least at one longitudinal end thereof; and
a retrieval catheter having a probe with an end which is ferromagnetic and is attracted to the head of the medical filter by magnetic force when proximate thereto.
   In accordance with the method of the present invention, a medical filter in a body passage is retrieved by use of magnetic force where the medical filter has a retrieval section with a head which is ferromagnetic by:
advancing a retrieval catheter having a probe with an end which is ferromagnetic through the body passage until the ferromagnetic end of the probe is proximate to said ferromagnetic head of said filter and is brought into alignment and contacting relationship therewith by said magnetic force;
withdrawing the ferromagnetic end of the probe and the medical filter into the retrieval catheter.

Further understanding of the present invention will be had from the following description taken in conjunction with the accompanying drawings and appended claims.

### BRIEF DESCRIPTION OF THE DRAWINGS

FIG. 1 is a perspective view, broken away, of a preferred embodiment of a system of the present invention, showing a medical filter and a retrieval catheter;

FIG. 2 is a side elevation view, broken away, of the medical filter and retrieval catheter of FIG. 1 in operative relationship and illustrating an initial step of carrying out a preferred method of the present invention;

FIG. 3 is a side elevation view, broken away, illustrating another step of the preferred method of FIG. 2 in which the medial filter and retrieval catheter come into contacting and attaching relationship;

FIG. 4 is a side elevation view, broken away, illustrating a further step of the preferred method of FIG. 2 in which the medical filter and retrieval catheter are brought into alignment;

FIG. 5 is a side elevation view, broken away, illustrating a further step of the preferred method of FIG. 2 in which the arms of the snaring device of the retrieval catheter are advanced over the head of the medical filter;

FIG. 6 is a side elevation view, broken away, illustrating the step of the preferred method of FIG. 2 in which the arms of the snaring device of the retrieval catheter are urged radially inwardly as they are brought into the sheath of the retrieval catheter;

FIG. 7 is a side elevation view, broken away, illustrating the step of the preferred method of FIG. 2 of pulling the filter into the retrieval sheath; and

FIG. 8 is a side elevation, broken away, showing an alternative preferred embodiment of a retrieval catheter of the present invention.

### DETAILED DESCRIPTION OF THE PREFERRED EMBODIMENTS

The following description of the preferred embodiments of the present invention is intended to be merely illustrative in nature, and as such, is not intended to limit in any way the present invention, its application, or uses. Numerous modifications may be made by those skilled in the art without departing from the true spirit and scope of the invention. For example, the present invention may be used in veins but is also useful in other body passages.

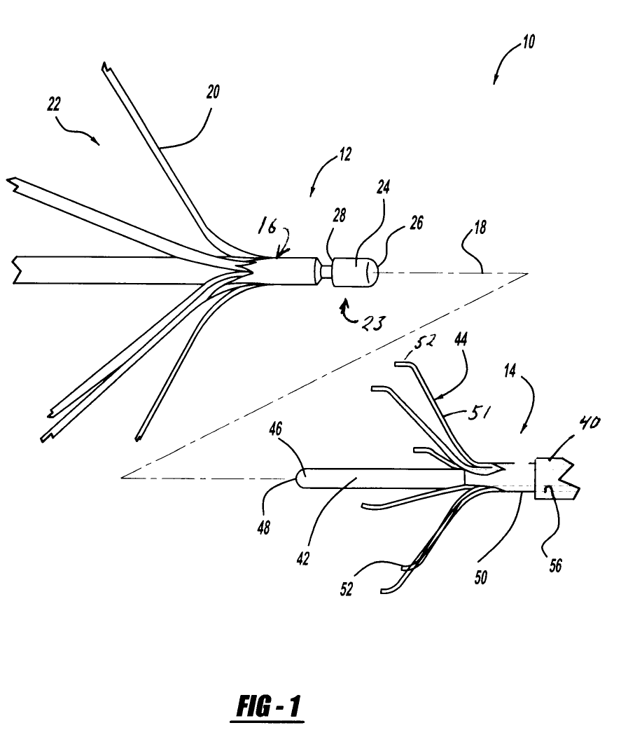

Now referring to FIG. 1, a preferred embodiment of a medical filter retrieval system of the present invention is shown and indicated generally by the numeral 10. System 10 comprises a medical filter 12 and a retrieval catheter 14 operatively associated therewith as will be further understood from the following description of this preferred embodiment of the present invention. Medical filter 12 and retrieval catheter 14 are designed to cooperate with each other to be self aligning and to facilitate using retrieval catheter 14 to remove filter 12 from a vein or other bodily passage where it has been implanted.

Medical filter 12 is made of a resilient material which tends to expand to the form illustrated in FIG. 1 but can be compressed to a smaller diameter form such as when inside a suitable delivery catheter or withdrawn into a retrieval catheter. Medical filter 12 has a body 16 with a longitudinal axis 18 and carries a plurality of radially and axially extending struts 20. The plurality of struts 20 define a filter basket 22. It will be appreciated by those skilled in the art that struts 20 and body 16 may be modified within the scope of the present invention and that body 16 may carry additional struts and have an opposing filter basket not shown in the figure. Filter 12 may be symmetrical or asymmetrical in form.

In accordance with the present invention, medical filter 12 has a retrieval section 23 at one end thereof. Retrieval section 23 is an elongated body having a longitudinally centered head 24 at its rightward end as shown in FIG. 1. Filter head 24 is characterized by having a ferromagnetic nature, a convex curved forwardly facing surface 26, and a rearward shoulder with a generally flat rearward facing surface 28. Filter head 24 thus has a generally semi-spherical shape, which term as used herein includes semi-spherical, semi-ellipsoidal, and semi-parabloid shapes, and provides forward facing surface 26.

Filter head 24 is provided with a ferromagnetic feature to cooperate with retrieval catheter 14 as described in more detail below. Thus, filter head 24 may be made of a ferromagnetic material or it may incorporate or encapsulate a ferromagnetic material. Rare earth magnetic materials such as samarium cobalt or neodymium iron boron are preferred magnetic materials because of their strong magnetic force relative to their size. The rare earth magnetic material may be encapsulated in traditional biocompatible materials in sufficient concentrations for sufficient magnetic force. Other suitable ferromagnetic materials for head 24 include, but are not limited to, duplex austenitic-ferritic stainless steel and ferritic stainless steel. Alternatively, head 24 may have a ferromagnetic material welded or glued thereon. Also alternatively, filter head 24 may be tubular with ferromagnetic material placed inside the tube.

The remainder of filter 12 may be made of any suitable material using a variety of methods. Suitable conventional materials and methods are well known in the art. Nitinol and stainless steel are examples of suitable materials but other materials may be used so long as the material has the desired characteristics of strength, resilience, flexibility, biocompatibility and endurance and is suitable for the particular manufacturing technique employed. It is, of course, required that the material employed be capable of expanding to the desired shape upon ejection from the delivery catheter and reduction in diameter when withdrawn into the lumen of the retrieval catheter. Thus, the material must also be sufficiently resilient to accomplish both compression and expansion.

Suitable methods of manufacture of body filter 12 include cutting a pattern into a tube to enable expansion of the tube into the desired body and struts. Another suitable method is forming the struts and body from separate strips or wires and then joining the respective parts together by suitable methods which are well known in the art.

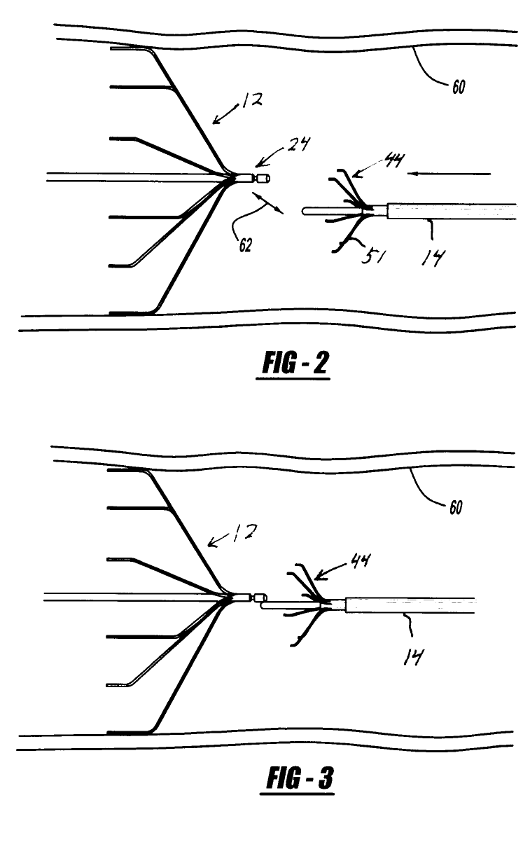

Retrieval catheter 14 has an elongated sheath 40 coaxially encompassing central probe 42 and snaring device 44. Probe 42 has a forward end 46 which is magnetically attracted to head 24 and which comprises a ferromagnetic material or a magnetic material. The forward face 48 of forward end 46 of probe 42 is a generally hemispherical surface 48 to facilitate alignment of forward end 46 with head 24 as described in more detail hereinafter. Snaring device 44 has a plurality of somewhat flexible arms 51 which are spring biased radially outwardly and which have reentrantly bent ends 52. Snaring device 44 has a tubular portion 50 which is coaxially located within lumen 56 of retrieval catheter 14. Tubular portion 50 is slidably located within lumen 56 so that, with respect to FIG. 2, it may be moved rightwardly with respect to sheath 40 to thereby draw arms 51 and ends 52 radially inwardly or it may be moved leftwardly with respect to sheath 40 to the position shown in FIG. 2 thereby allowing arms 51 and ends 52 to expand radially outwardly.

Having described a preferred system of the present invention, further understanding of the unique character and advantages of the present invention will be had by an understanding of the method for retrieval of filter 12. It will be further appreciated that the present method relates to a method for retrieval of a medical filter which has been previously position within a vein or body passage. For example and not by way of limitation, filter 12 is intended to be initially deployed in the lumen of a delivery catheter as is conventional in the art. Insertion of filter 12 into a delivery catheter can be by any conventional method including by simply pushing filter 12 into the lumen at the distal end of a delivery catheter. Alternatively, filter 12 may be inserted into the proximal end of a delivery catheter and pushed by means of a push wire or the like to the distal end of the catheter. Emplacement of filter 12 in a vein or other body passage may also be done by conventional means well known in the art. For example, a delivery catheter carrying a radially compressed filter 12 may be inserted along a body vessel in a patient until its distal end is near the desired site for treatment. The delivery catheter may be inserted using either a femoral approach or a jugular approach. A push wire may be used to eject filter 12 from the lumen of the delivery catheter by pushing filter 12. As filter 12 is ejected, arms 20 expand radially outwardly until contacting the walls of the vessel.

Once emplaced in a body passage such as a vein, in many cases there will be no desire to retrieve filter 12 from the vessel and there will be no need for the present invention. However, is some cases there will be a desire to retrieve filter 12 from the vessel and is such cases the present invention provides an advantageous system and method for this purpose. Now referring to FIGS. 2-9, further understanding can be had of the method of the present invention using the system of the present invention.

FIG. 2 illustrates the initial step of the method of retrieving filter 12 in accordance with this invention. Filter 12 is shown in place in vessel 60. Retrieval catheter 14 is shown having been advanced through vessel 60 to a location proximate to filter 12. As will be appreciated by the skilled artisan, such advancement may be from either a femoral or jugular approach but must be made toward an appropriate head 24 of medical filter 12. If medical filter 12 has a head 24 at either of its longitudinal ends, then medical filter 12 can be approached from either direction. Of course, if medical filter 12 has a head 24 at only one end, then it must be approached toward the end that has head 24. Snare 44 is preferably withdrawn into lumen 56 of retrieval catheter 14 during advancement of catheter 14 through vessel 60 to facilitate the advancement of the catheter through the vessel. However, when proximate to medical filter 12, tubular portion 54 of snaring device 44 is extended beyond sheath 40 of retrieval catheter 14 (leftwardly as viewed in FIG. 2) to allow arms 51 to flex radially outwardly into position to capture head 24 of medical filter 12 for retrieval thereof. As illustrated in FIG. 2, head 24 of medical filter 12 and forward end 46 of probe 42 are attracted to each other by magnetic force 62 and as retrieval system 14 is advanced toward medical filter 12, magnetic force 62 urges forward face 48 of end 46 of probe 42 into contacting relationship with forward facing surface 26 of head 24. This contacting relationship is shown in FIG. 3 and represents the step of probe 42 magnetically attaching to medical filter 12.

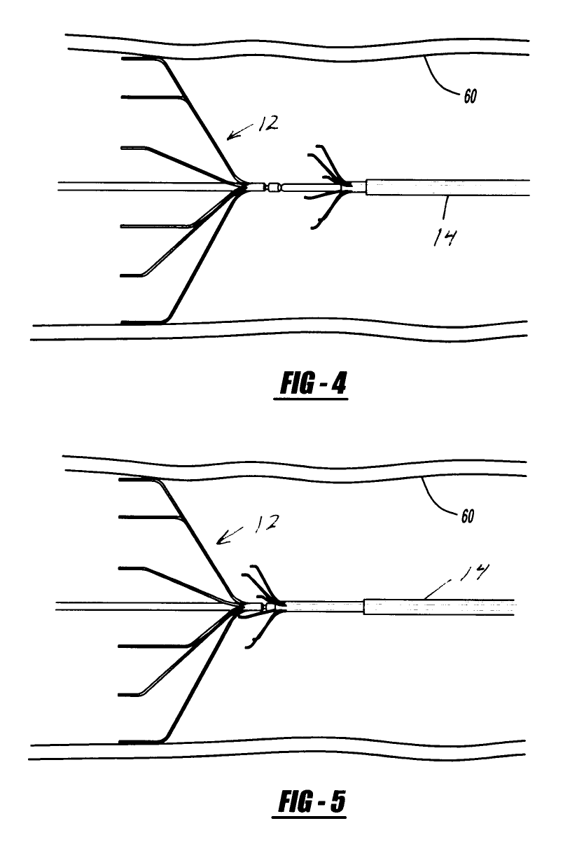

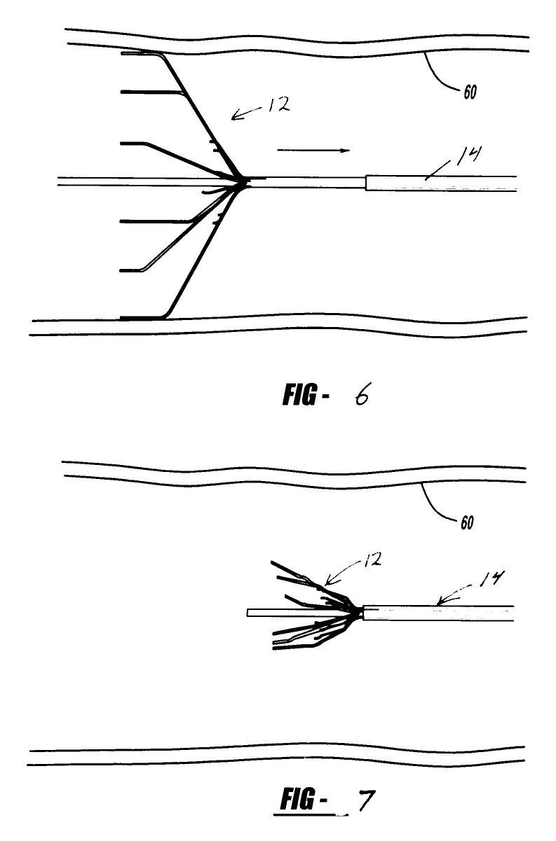

It is contemplated that head 24 and probe 42 will not be initially in exact alignment but will be nearly aligned as shown in FIG. 3. Substantial alignment or centering of head 24 and probe 42 as shown in FIG. 4 can be achieved by pulling rightwardly as shown in FIG. 4 to utilize the round ends of each surface 26 and 48 to align the axis of probe 42 with the axis of medical filter 12. Then snaring device 44 can be advanced to the left as viewed in FIGS. 5 and 6.

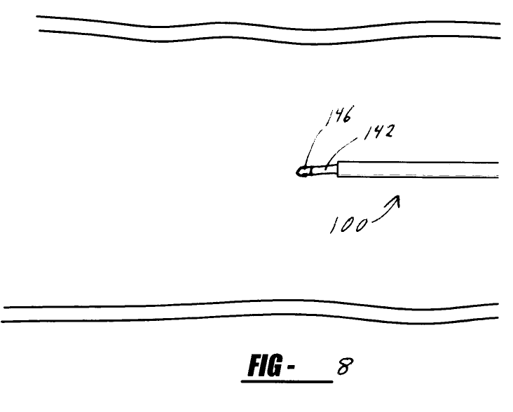

As shown in FIG. 6, after probe 42 and head 24 are aligned, coaxial snaring device 44 is advanced, if necessary, leftward as shown in the FIG. 6, so that hooks 52 are positioned radially outwardly from shoulder 28 of head 24. Then, tubular portion 54 of snaring device 44 is manipulated rightwardly with respect to sheath 40 which urges arms 50 radially inwardly and into neck portion 29 of medical filter 12 as shown in FIG. 7.

FIG. 7 illustrates the step of pulling medical filter 12 into lumen 56 of retrieval catheter 14 by pulling snaring device into lumen 56. Hooks 52 abut against shoulder 28 when retrieval catheter 14 is manipulated rightwardly and struts 20 are compressed radially inwardly by sheath 40 as medical filter 12 is pulled into lumen 56. Finally, FIG. 9 illustrates the step of retrieving catheter 14 which now encompasses medical filter 12 from vessel 60.

Now referring to FIG. 8, an alternative preferred embodiment of a retrieval catheter of the present invention is shown and indicated generally by the numeral 100. Retrieval catheter 100 is generally analogous to retrieval catheter 14, but does not have snaring device 44. Thus, retrieval catheter 100 carries probe 142 which has forward end 146 which is comprised of a magnetic material. The magnetic material of end 146 serves to align probe 142 with head 24 of medical filter 12 and is characterized by a magnetic force at least sufficient to remain attached to head 24 while probe 142 is withdrawn into the sheath of retrieval catheter 100.

While preferred embodiments of the present invention have been specifically described above, it will be appreciated by those skilled in the art that the present invention is subject to variations and modifications. For example, the filter may be cut from a single tube and have end rings which are integral, i.e., one piece, with the spine and struts. The filter may have wire struts and end rings which are tubular.

What is claimed is:

1. A medical filter retrieval system for retrieval of a medial filter from placement inside a body passage of a patient, comprising:
a medical filter having an elongated body with a ferromagnetic head at one end thereof;
a retrieval catheter having a lumen with an elongated central probe therein, said probe located coaxially within said catheter and being longitudinally movable therein, said probe having a forward end portion characterized by comprising a ferromagnetic material; and
said forward end portion of said probe being attracted to said ferromagnetic head when proximate thereto by a magnetic force.

2. The medical filter retrieval system of claim 1, wherein at least one of said ferromagnetic head and said forward end portion of said probe comprises a rare earth magnetic material.

3. The medical filter retrieval system of claim 1, wherein said retrieval catheter has a snaring device.

4. The medical filter retrieval system of claim 3, wherein said snaring device has a tubular portion located coaxially within said lumen and has a forward end with a plurality of arms having hooks on each end thereof.

5. The medical filter retrieval system of claim 1 wherein said ferromagnetic head of said medical filter and said forward end portion of said probe each have a semi-spheroidal shape.

6. The medical filter retrieval system of claim 1 wherein at least one of said ferromagnetic head and said forward portion of said probe has a layer of ferromagnetic material thereon.

7. The medical filter retrieval system of claim 1 wherein at least one of said ferromagnetic head and said forward portion of said probe comprises a ferromagnetic material homogeneously formed therewith.

8. The medical filter retrieval system of claim 3 wherein said ferromagnetic head of said medical filter and said forward end portion of said probe each have a semi-spheroidal shape.

9. A medical filter retrieval system for retrieval of a medial filter from placement inside a body passage of a patient, comprising:
a medical filter having an elongated body with a ferromagnetic head at one end thereof, said head comprising a rare earth magnetic material and having a forward-facing convex surface;
a retrieval catheter having a lumen with an elongated central probe therein, said probe located coaxially within said catheter and being longitudinally movable therein, said probe having a forward end portion characterized by comprising a rare earth ferromagnetic material and a forward-facing convex surface;
a snaring device coaxially located in said lumen of said retrieval catheter and having a forward end with a plurality of arms each biased radially outwardly and each having a re-entrently bent end;
said forward end portion of said probe being attracted by a magnetic force to said ferromagnetic head when proximate thereto.

10. A method of retrieving a medical filter from a body passage, said medical filter having a ferromagnetic head, said method comprising advancing a ferromagnetic probe in said body passage into magnetic contacting relationship with said ferromagnetic head and retrieving said probe and said head while maintaining said contacting relationship.

11. The method of retrieving a medical filter as in claim 10, wherein each said probe and said head have an outward facing convex surface in contacting relationship, wherein said probe and said head are further aligned by manipulating said probe in a direction slightly away from said medical filter.

12. The method of retrieving a medical filter of claim 10, wherein said ferromagnetic probe is coaxially located within a lumen of a catheter sheath and said probe is advanced by advancing said catheter sheath in said body passage.

13. The method of retrieving a medical filter of claim 12, wherein said body passage is a vein.

14. The method of retrieving a medical filter of claim 11, wherein said probe is coaxially located in a lumen of a retrieval catheter which carries a snaring device, said retrieving step being carried out by engaging said snaring device with said head.

15. The method of retrieving a medical filter of claim 15, wherein said snaring device has a plurality of arms each biased radially outwardly and having a re-entrently bend end and said head of said medical filter having a rearward facing shoulder, said retrieving being carried out by urging said re-entrently bent ends of said arms into interfering relationship with said shoulder.

16. The method of retrieval of a medical filter comprising the steps of:
providing a medical filter inside a body passage of a patient, said filter comprising:
in a radially compressed state, a longitudinally extending spine having a plurality of struts secured at one longitudinal end of said spine and a plurality of struts secured at an opposite longitudinal end of said spine, each of said struts extending generally longitudinally along said spine and each of said struts being biased in a radially outward direction; and
in a radially expanded state, each of said struts extending in a direction generally radially outwardly and axially from said spine and each of said struts having a first bend proximate to said spine in a direction away from said spine and a second bend at each free end portion in a direction toward said spine; and
withdrawing said filter into a lumen of a catheter by contacting each strut secured at one end of said spine with a distal tubular end of said catheter and bending each said strut radially inwardly while withdrawing a first part of said filter into said lumen; and then
continuing to withdraw said filter into said lumen by contacting each strut secured at said other end of said spine with said distal tubular end of said catheter thereby bending said struts in an inversely to a radially inward position as said filter is withdrawn into said lumen.
